(19) United States
(12) Patent Application Publication
(10) Pub. No.: US 2011/0118517 A1
(43) Pub. Date: May 19, 2011

(54) Manufacturing Process for Branched and Linear Alkylated Benzene as Precursor for Enhanced Oil Recovery Surfactant

(75) Inventors: Stephen W. Sohn, Arlington Heights, IL (US); Mark G. Riley, Hinsdale, IL (US); Bryan K. Glover, Algonquin, IL (US)

(73) Assignee: UOP LLC, Des Plaines, IL (US)

(21) Appl. No.: 12/622,020

(22) Filed: Nov. 19, 2009

(51) Int. Cl. C07C 5/00

(52) U.S. Cl. 585/256; 585/323

(57) ABSTRACT

A process is presented for the preparation of surfactants that are useable in enhanced oil recovery. The surfactants are long chained sulfonated alkylaryl compounds. The process includes recovering linear and lightly branched paraffins from a hydrocarbon stream, dehydrogenating the paraffins, and then alkylating benzene with the olefins generated. The process uses pentasil zeolites to selectively separate the normal and lightly branched paraffins from the hydrocarbon stream.

### FIELD OF THE INVENTION

This invention relates to the field of sulfonated alkyl benzenes. In particular, the invention relates to the selective production of high molecular weight alkyl benzenes for the production of surfactants.

### BACKGROUND OF THE INVENTION

Alkylation of benzene produces alkylbenzenes that may find various commercial uses, e.g., alkylbenzenes can be sulfonated to produce surfactants, for use in detergents. In the alkylation process, benzene is reacted with an olefin the desired length to produce the sought alkylbenzene. The alkylation conditions comprise the presence of homogeneous or heterogeneous alkylation catalyst such as aluminum chloride, hydrogen fluoride, or zeolitic catalysts and elevated temperature.

Various processes have been proposed to alkylate benzene. One commercial process involves the use of hydrogen fluoride as the alkylation catalyst. The use and handling of hydrogen fluoride does provide operational concerns due to its toxicity, corrosiveness and waste disposal needs. Solid catalytic processes have been developed that obviate the need to use hydrogen fluoride. Improvements in these solid catalytic processes are sought to further enhance their attractiveness through reducing energy costs and improving selectivity of conversion while still providing an alkylbenzene of a quality acceptable for downstream use such as sulfonation to make surfactants.

For detergent alkylation, alkylbenzenes for making sulfonated surfactants must be capable of providing a sulfonated product of suitable clarity, biodegradability and efficacy. However, for enhanced oil recovery, the criteria for a suitable product are different from the commercial requirements for detergents. The use of heavier sulfonated surfactants in enhanced oil recovery center more on the solubility considerations, and the surfactants will usually be subsequently processed with the recovered oil, but some will remain in the formation holding the oil. Therefore, the biodegradability is not important, but the ability to solubilize heavy oil is more important.

Improvements in the catalysts have facilitated the production of linear alkylbenzenes, as shown in U.S. Pat. No. 6,133,492, U.S. Pat. No. 6,521,804, U.S. Pat. No. 6,977,319, and U.S. Pat. No. 6,756,030. However, the limitation to linear alkylbenzenes increases the price pressure on detergents and there is a need to expand the availability of materials that can be used in detergents.

### SUMMARY OF THE INVENTION

The present invention comprises a process for the production of alkylbenzenes. The process is for the selective separation of normal and lightly branched paraffins having 14 to 23 carbons from a hydrocarbon mixture. The lightly branched paraffins are monomethyl branched paraffins. The hydrocarbon stream is passed through an adsorption separation system wherein the normal and lightly branched paraffins are selectively adsorbed, and then extracted. The extracted normal and lightly branched paraffins are passed through a dehydrogenation reactor to selectively dehydrogenate the paraffins to an olefin rich stream. The olefins are passed, along with a benzene feedstream, to an alkylation reactor to generate an alkylbenzene product stream.

The process utilizes a pentasil zeolite to preferentially allow for normal and monomethyl paraffins to be adsorbed in the adsorption separation system. The pentasil zeolites include at least one from the following: ZSM-5, As—Si—O-MFI, Fe—Si—O-MFI, Ga—Si—O-MFI, AMS-1B, AZ-1, Bor-C, Boralite C, Encilite, FZ-1, LZ-105, Monoclinic HZSM-5, NU-4, NU-5, Silicalite, TS-1, TSZ, TSZ-III, TZ-01, USC-4, USI-108, ZBH, ZKQ-1B, ZMQ-TB, and organic-free ZSM-5.

The process can further comprise selective hydrogenation of the olefin rich stream to remove reactive diolefins and acetylenes to provide an enriched olefin stream for benzene alkylation. The process can further include the sulfonation of the alkylbenzene product stream to produce a surfactant product.

Other objects, advantages and applications of the present invention will become apparent to those skilled in the art from the following detailed description.

### DETAILED DESCRIPTION OF THE INVENTION

The present invention is a process for recovering lightly branched and normal paraffins for use in the production of detergents. The paraffins selected are large and are recovered from light gas oil. Currently, only normal paraffins are produced using UOP's Molex™ technology. However, with the development of detergent technology, lightly branched paraffins are found to be as effective, and as biodegradable as normal paraffins used in detergents. The present invention uses sorbex technology for recovering mono-methyl paraffins in the C14 to C23 range. The availability of alkylbenzenes having large alkyl groups is limited.

The typical products from an atmospheric crude distillation unit (operated at 5-10 psig) are shown in Table 1. Light gas oil provides for a hydrocarbon mixture that includes many components, of which the desired C14 to C23 paraffins are included.

TABLE 1
typical product streams

| product stream | temp. cut range, ° F. | temp. cut range, ° C. |
|---|---|---|
| full range naphtha | gas to 380 | gas to 193 |
| kerosene | 380 to 480 | 193 to 249 |
| light gas oil | 480 to 610 | 249 to 321 |
| heavy gas oil | 610 to 690 | 321 to 366 |
| fuel oil | +690 | +366 |

Sulfonate alkylated benzenes are useful surfactants in Enhanced Oil Recovery operations. Literature indicates that heavier paraffins (C14-23) make better alkyl group for this surfactant. Limited branching and phenyl group added to the center of the alkyl group also improve surfactant performance. Unfortunately, large commercial quantities of lightly branched paraffins are not available.

Light gas oil is lightly hydrotreated, and then passed to a sorbex unit for separation of normal and mono-methyl branched paraffins from the light gas oil. A sorbex unit is an adsorption separation unit, using simulated moving bed technology to separate components in a mixture. Simulated moving bed technology is a continuous process, and is described in U.S. Pat. No. 2,985,589 (Broughton et al.), and is incorporated by reference in its entirety.

The present invention is a process for the production of lightly branched alkyl-benzenes. The process includes passing a hydrocarbon mixture having paraffins with 14 to 23 carbon atoms through an adsorption separation system. The hydrocarbon mixture comes from a light cycle oil, or a product stream from a distillation unit having a temperature cut between 480° F. to 610° F. The separation process generates an extract stream comprising normal and monomethyl branched paraffins, and a raffinate stream comprising non-normal and more highly branched paraffins. The extract stream is passed to a dehydrogenation reactor, where the paraffins are converted to olefins, with a small amount of diolefins and acetylenes. The olefin stream is passed with an aromatic stream to an alkylation reactor to produce a product stream comprising alkylaromatic compounds. The extraction process utilizes an adsorbent to allow for more lightly branched paraffins, and is one that is chosen to be more tolerant of adsorbent poisons.

The hydrocarbon mixture is a lightly hydrotreated light gas oil. The light hydrotreating contributes to reducing the naphthenic content and partial saturation of aromatics with ring opening. This provides a greater paraffin and olefin content for further processing.

The hydrocarbon mixture is then passed to an adsorption separation system using the simulated moving bed technology. The adsorption separation system comprises an adsorbent comprising a pentasil molecular sieve having larger pores to accommodate the larger olefins, including the methyl branched olefins. The pentasil molecular sieves include pentasil zeolites. Pentasil zeolites for use in this process include ZSM-5, As—Si—O-MFI, Fe—Si—O-MFI, Ga—Si—O-MFI, AMS-1B, AZ-1, Bor-C, Boralite C, Encilite, FZ-1, LZ-105, Monoclinic HZSM-5, NU-4, NU-5, Silicalite, TS-1, TSZ, TSZ-III, TZ-01, USC-4, USI-108, ZBH, ZKQ-1B, ZMQ-TB, organic-free ZSM-5, and mixtures thereof. A preferred adsorbent is silicalite, where the adsorbent has pores of sufficient size to admit normal paraffins and mono-methyl paraffins. The adsorbent can also be a mesoporous silica adsorbent. The separation process produces an extract stream comprising normal and mono-methyl paraffins, and a raffinate stream comprising the remainder of the hydrocarbons.

The extract stream is passed to a selective dehydrogenation unit where the paraffins are dehydrogenated to generate an olefin stream. The dehydrogenation process, in addition to producing olefins, generates small amounts of diolefins and acetylenes from the paraffins. Optionally, the olefin stream is further processed to reduce the diolefin and acetylene content. The olefin stream is passed to a hydrogenation reactor where the diolefins and acetylenes are selectively hydrogenated to increase the olefin content of the olefin stream, creating an enriched olefin stream.

The enriched olefin stream is passed to an alkylation reactor, along with an aromatic stream for alkylation. The aromatic stream is preferably benzene. The alkylation reactor comprises an alkylation catalyst for performing the alkylation of benzene with the olefins. Alkylation catalysts include aluminum chloride, hydrogen fluoride, or zeolitic catalysts. Zeolitic catalysts include acidic zeolites having large pores and super cages for allowing access of both the aromatic compound and olefin, and still having space for the compounds to react.

The process can further include the process of passing the enriched olefin stream through a separation process for removing aromatics produced in the dehydrogenation reactor, thereby generating an aromatics free, or aromatics reduced enriched olefin stream. The aromatics to be removed are the non-benzene aromatics, including toluene, xylenes, and aromatics with one or more small chained alkyl groups attached. Examples of small chained alkyl groups would be alkyl groups containing 8 or less carbon atoms, and can even contain 12 or less carbon atoms when they alkyl groups are highly branched, or the alkylaryl compounds are poly alkylated. The aromatics reduces enriched olefin stream is then passed to the alkylation reactor to generate the desired long chained monoalkylbenzene. The monoalkylbenzene is then sulfonated to generate a surfactant that is useable in enhanced oil recovery.

In one embodiment, the present invention is a process for the production of lightly branched alkyl-benzenes comprising passing a hydrocarbon mixture from a light gas oil cut of petroleum distillation through an adsorption separation system. An extract stream comprising normal and monomethyl branched paraffins is generated, and a raffinate stream comprising non-normal and more highly branched paraffins is recovered. The extract stream is passed to a dehydrogenation reactor to create an olefin stream. The olefin stream includes olefins and diolefins, and small amounts of aromatics that are larger than benzene. The olefin stream is passed to a separation unit to remove the aromatic compounds which would have a deleterious affect on product quality, and thereby creates a purified olefin stream. The purified olefin stream is passed to an alkylation reactor, along with a benzene feedstream, to create an alkylbenzene product stream.

The adsorption separation system uses an adsorbent having larger pores to accommodate the larger hydrocarbons, but not too large of pores to limit the amount of non-normal and highly branched hydrocarbons adsorbed. A preferred adsorbent is a pentasil zeolite, and includes one or more zeolites such as: ZSM-5, As—Si—O-MFI, Fe—Si—O-MFI, Ga—Si—O-MFI, AMS-1B, AZ-1, Bor-C, Boralite C, Encilite, FZ-1, LZ-105, Monoclinic HZSM-5, NU-4, NU-5, Silicalite, TS-1, TSZ, TSZ-III, TZ-01, USC-4, USI-108, ZBH, ZKQ-1B, ZMQ-TB, organic-free ZSM-5.

The alkylbenzene product stream is further processed through sulfonation of the alkylbenzene product stream to create the surfactant product.

While the invention has been described with what are presently considered the preferred embodiments, it is to be understood that the invention is not limited to the disclosed embodiments, but it is intended to cover various modifications and equivalent arrangements included within the scope of the appended claims.

1. A process for the production of lightly branched alkyl-benzenes comprising:
passing a hydrocarbon mixture comprising paraffins having from 14 to 23 carbon atoms through an adsorption separation system, thereby generating an extract stream comprising normal and monomethyl branched paraffins, and a raffinate stream comprising non-normal and more highly branched paraffins;
passing the extract stream to a dehydrogenation reactor thereby creating an olefin stream comprising olefins, and diolefins;
passing the olefin stream and an aromatic stream to an alkylation reactor, thereby creating an alkylaromatic stream.

2. The process of claim 1 wherein the hydrocarbon mixture is a lightly hydrotreated light gas oil.

3. The process of claim 1 wherein the adsorption separation system comprises a simulated moving bed adsorption separation system.

4. The process of claim 1 wherein the adsorption separation system utilizes silicalite for the adsorbent.

5. The process of claim 1 wherein the adsorbent in the adsorption separation system is a mesoporous silica adsorbent.

6. The process of claim 1 further comprising passing the olefin stream comprising olefins and diolefins to a selective hydrogenation reactor to reduce the diolefin content of the olefin stream.

7. The process of claim 1 wherein the adsorbent in the adsorption separation system comprises a pentasil molecular.

8. The process of claim 7 wherein the adsorbent in the adsorption separation system comprises a pentasil zeolite.

9. The process of claim 8 wherein the pentasil zeolite is selected from the group consisting of ZSM-5, As—Si—O-MFI, Fe—Si—O-MFI, Ga—Si—O-MFI, AMS-1B, AZ-1, Bor-C, Boralite C, Encilite, FZ-1, LZ-105, Monoclinic HZSM-5, NU-4, NU-5, Silicalite, TS-1, TSZ, TSZ-III, TZ-01, USC-4, USI-108, ZBH, ZKQ-1B, ZMQ-TB, organic-free ZSM-5, and mixtures thereof.

10. The process of claim 9 wherein the adsorbent in the adsorption separation system comprises silicalite.

11. The process of claim 1 further comprising:
passing the olefin stream through a separation process to create an olefins stream having a reduced aromatics content; and
passing the olefins stream with the reduced aromatics content to the alkylation reactor.

12. The process of claim 1 further comprising sulfonating the alkylaromatic stream.

13. A process for the production of lightly branched alkyl-benzenes comprising:
passing a hydrocarbon mixture from a light gas oil cut of petroleum distillation through an adsorption separation system, thereby generating an extract stream comprising normal and monomethyl branched paraffins, and a raffinate stream comprising non-normal and more highly branched paraffins;
passing the extract stream to a dehydrogenation reactor thereby creating an olefin stream comprising olefins, and diolefins;
passing the olefin stream to a separation unit to remove aromatic compounds from the olefin stream, thereby creating a purified olefin stream; and
passing the purified olefin stream and an aromatic stream to an alkylation reactor, thereby creating an alkylbenzene product stream.

14. The process of claim 13 wherein the light gas oil cut is a petroleum atmospheric distillation fraction having a boiling point range between 249 to 321 C.

15. The process of claim 13 wherein the aromatic stream is benzene.

16. The process of claim 13 wherein the adsorbent for the adsorption separation system comprises a pentasil zeolite.

17. The process of claim 16 wherein the pentasil zeolite is selected from the group consisting of ZSM-5, As—Si—O-MFI, Fe—Si—O-MFI, Ga—Si—O-MFI, AMS-1B, AZ-1, Bor-C, Boralite C, Encilite, FZ-1, LZ-105, Monoclinic HZSM-5, NU-4, NU-5, Silicalite, TS-1, TSZ, TSZ-III, TZ-01, USC-4, USI-108, ZBH, ZKQ-1B, ZMQ-TB, organic-free ZSM-5, and mixtures thereof.

18. The process of claim 13 further comprising sulfonating the alkylaromatic product stream.

19. A process for the production of lightly branched alkyl-benzenes comprising:
passing a hydrocarbon mixture from a light gas oil cut of petroleum distillation through an adsorption separation system, thereby generating an extract stream comprising normal and monomethyl branched paraffins, and a raffinate stream comprising non-normal and more highly branched paraffins;
passing the extract stream to a dehydrogenation reactor thereby creating an olefin stream comprising olefins, and diolefins;
passing the olefin stream to a separation unit using a pentasil zeolite for the adsorbent in the separation unit to remove aromatic compounds from the olefin stream, thereby creating a purified olefin stream;
passing the purified olefin stream and an aromatic stream to an alkylation reactor, thereby creating an alkylbenzene product stream; and
sulfonating the alkylaromatic product stream.
